(19) United States
(12) Patent Application Publication
(10) Pub. No.: US 2022/0280040 A1
(43) Pub. Date: Sep. 8, 2022

(54) SYSTEM AND METHOD FOR CONTINUOUS MONITORING OF RESPIRATORY AILMENTS

(71) Applicant: RESMED CORP., SanDiego, CA (US)

(72) Inventors: FAIZAN JAVED, Bella Vista, New South Wales (AU); DAVID MATTHEW KEARNS, Bella Vista, New South Wales (AU); CIARA ANNE O'DWYER, Dublin 2 (IE); DAVID CRIMMINS, Dublin 2 (IE); SARAH TERESE MCGANN, Sydney (AU); JOSE RICARDO DOS SANTOS, San Diego, CA (US)

(21) Appl. No.: 17/631,728

(22) PCT Filed: Jul. 31, 2020
(86) PCT No.: PCT/US20/44632

§ 371 Date: Jan. 31, 2022

Related U.S. Application Data
(60) Provisional application No. 62/881,330, filed on Jul. 31, 2019.
(60) Provisional application No. 62/941,185, filed on Nov. 27, 2019.

(51) Int. Cl. A61B 5/00; A61B 5/0205; A61B 5/08; G16H 10/60; G16H 50/20; G16H 50/30

(52) U.S. Cl. CPC A61B 5/0022; A61B 5/0205; A61B 5/0809; A61B 5/0816; A61B 5/7264; A61B 5/7275; G16H 10/60; G16H 50/20; G16H 50/30; A61B 5/6802; A61B 5/746; A61B 2560/0242

(57) ABSTRACT

A system and method to determine symptoms of respiratory ailments is disclosed. The system includes a transceiver operable to receive data from a monitor attached to a patient. The monitor includes a plurality of sensors, each of the plurality of sensors outputting physiological data related to respiration of the patient. An analytics platform is coupled to the transceiver to analyze the physiological data to determine the occurrence of a symptom of a respiratory condition, disorder or ailment in the patient.

### PRIORITY CLAIM

This application claims priority to and benefit of U.S. Provisional Patent Application No. 62/881,330, filed on Jul. 31, 2019 and U.S. Provisional Patent Application No. 62/941,185, filed on Nov. 27, 2019, each of which is hereby incorporated by reference herein it its entirety.

### TECHNICAL FIELD

The present disclosure relates generally to disease detection systems, and more specifically to a continuous monitoring system for respiratory ailments such as asthma.

### BACKGROUND

Many people suffer from respiratory ailments such as asthma or chronic obstructive pulmonary disease (COPD). For example, asthma is a common, chronic respiratory condition that causes airways to narrow, making it difficult to breathe. Additionally, asthma may cause wheezing, chest tightening, shortness of breath, and coughing. Asthma may be caused by an oversensitivity to inhaled substances that causes the bronchial airways to constrict and tighten. The airways may also swell and secrete mucous, further constricting airflow. During asthma attacks, the airways may narrow to the point where the condition may be life threatening.

In the United States alone, over 25 million people suffer from asthma, 7 million of which are children. Asthma has no cure, but may be managed with inhaled medications. Some patients may even eliminate most symptoms of asthma with regular usage of medication. Generally, asthma medications may be broken down into two categories: daily preventive treatments and rescue medications. Rescue medications are generally bronchodilators that quickly relax the smooth muscle in the bronchioles in order to dilate the airways and improve ease of breathing during an asthma attack. Daily preventive treatments typically include anti-inflammatory drugs such as steroids that reduce the swelling and mucus production in the airways and accordingly reduce a patient's susceptibility to triggers. Preventive anti-inflammatories are effective at controlling and even preventing asthma symptoms.

Once asthma is diagnosed, patients may be prescribed the preventive anti-inflammatories that may be self-administered by an inhaler device. However, such treatments rely on early detection of asthma. Currently, there is no continuous monitoring of a patient to predict asthma attacks and therefore apply preventive treatments as symptoms appear. Health care providers must rely on patients appearing in person for periodic checkups. Health care professionals typically use a stethoscope to detect abnormal breathing during the checkup. Thus, impending asthma attacks may go undetected and become more severe thus increasing the likelihood that preventive treatments will be too late and rescue medications will be required.

There is a need for a system that allows for continuous monitoring of respiratory conditions, disorders, or ailments such as asthma to determine symptoms of such conditions. There is also a need for a system that includes a monitor that can continuously sense multiple physiological signals such as respiration rate, heart rate, breath shape, breath sound, tidal volume and others to predict a respiratory event such as an asthma attack or exacerbation. There is also a need for a system that provides an easy to use body monitor that may provide around the clock monitoring for respiratory conditions, disorders, or ailments.

### SUMMARY

The disclosed respiratory ailment monitoring system provides continuous measurements of signals relevant to respiratory conditions, disorders, or ailments. The disclosed system allows nighttime monitoring. The system includes an easy-to-use monitor having multiple types of sensors to determine data relevant to monitoring respiratory conditions, disorders, or ailments. Based on such data, the system may determine symptoms of respiratory ailments and predict respiratory events such as asthma attacks.

One disclosed example is a system to determine symptoms of respiratory ailments. The system includes a transceiver operable to receive data from a monitor attached to a patient. The monitor includes a plurality of sensors, each of the plurality of sensors outputting physiological data related to respiration of the patient. An analytics platform is coupled to the transceiver to analyze the physiological data to determine the occurrence of a symptom of a respiratory condition, disorder or ailment in the patient.

A further implementation of the example system is where the plurality of sensors includes a heart rate sensor and a respiratory sensor. Another implementation is where the system includes a portable computing device that receives the physiological data from the transceiver and transmits the physiological data to the analytics platform. Another implementation is where the analytics platform analyzes environmental data related to the patient in determining the occurrence of the symptom of the respiratory condition. Another implementation is where the analytics platform analyzes demographic data related to the patient in determining the occurrence of the symptom of the respiratory condition. Another implementation is where the plurality of sensors further includes an accelerometer. Another implementation is where the plurality of sensors further includes a pressure sensor. Another implementation is where the symptom is shortness of breath. Another implementation is where the analytics platform is configured to determine shortness of breath using a combination of: breathing effort determined from the pressure sensor and the accelerometer; and respiration rate determined from the respiratory sensor. Another implementation is where the plurality of sensors includes an audio sensor. Another implementation is where the analytics platform differentiates between a soft wheeze and other adventitious signals based on data from the audio sensor. Another implementation is where the analytics platform is executed on a remote server. Another implementation is where the analytics platform is configured to apply a model to the physiological data to determine the occurrence of a symptom of the respiratory condition. Another implementation is where the model is configured by machine learning based on collected physiological data and respiratory condition outcome data. Another implementation is where the analytics platform determines an occurrence of a symptom based on population health factors relevant to the patient. Another implementation is where the population health factors comprise social determinants of health. Another implementation is where the analytics platform infers the social determinants of health based on the geographic location of a home of the patient. Another implementation is where the population health factors comprise data gathered from another patient in a cohort of patients that is similar to the patient. Another implementation is where the analytics platform analyzes the physiological data to determine a risk evaluation of an event of the respiratory condition of the patient. Another implementation is where the analytics platform compares the risk evaluation with a threshold to predict the respiratory event. Another implementation is where the analytics platform initiates a corrective action in response to the predicted respiratory event. Another implementation is where the plurality of sensors includes an impedance plethysmography sensor. Another implementation is where the analytics platform determines the risk evaluation by: correlating impedance measurements from the impedance plethysmography sensor with lung volume; constructing a flow-volume curve from the lung volume; extracting one or more tidal volume parameters from the flow-volume curve; deriving features from the tidal volume parameters; and applying a model to the features to determine the risk evaluation. Another implementation is where the plurality of sensors includes an ECG sensor. Another implementation is where the analytics platform reject snoise generated by cardiac activity from the impedance measurements using the ECG sensor. Another implementation is where the plurality of sensors includes an accelerometer. Another implementation is where the analytics platform rejects movement artefacts from the impedance measurements using the accelerometer. Another implementation is where the one or more tidal volume parameters are drawn from the group consisting of: Time to Peak Expiratory Flow over Expiratory Time; Volume at Peak Expiratory Flow over Expiratory Tidal Volume; and Slope of post-peak Expiratory Flow Curve. Another implementation is where the model is configured by machine learning based on collected physiological data and respiratory condition outcome data.

Another disclosed example is a continuous monitoring device attachable to a patient. The monitoring device includes an enclosure having a surface that may be adhered to the patient. The monitoring device includes a plurality of sensors, each of the plurality of sensors continuously sensing different physiological data from the patient relating to a respiratory condition, disorder or ailment of the patient. A memory stores the physiological data. A transceiver transmits the sensed data to an external device.

A further implementation of the example monitoring device is where the plurality of sensors includes a heart rate sensor and a respiratory sensor. Another implementation is where the respiratory sensor is an impedance plethysmography sensor. Another implementation is where the monitor includes a pair of electrode pads configured to sense a voltage between the electrode pads. Another implementation is where the heart rate sensor is coupled to the pair of electrode pads. Another implementation is where the impedance plethysmography sensor is coupled to the pair of electrode pads. Another implementation is where the monitor includes a second pair of electrode pads to which the impedance plethysmography sensor is coupled for injection of low-amplitude, high-frequency current. Another implementation is where the enclosure has a form factor that is one of the group consisting of: a patch, a wristband, a necklace, and a vest. Another implementation is where the plurality of sensors includes an audio sensor. Another implementation is where the plurality of sensors includes an accelerometer and a gyroscope. Another implementation is where the plurality of sensors further comprises a pressure sensor. Another implementation is where the enclosure is fabricated from a flexible compliant material.

Another example is a system to monitor a respiratory condition of a patient. The system includes a monitor attachable to the patient. The monitor includes a plurality of sensors, each of the plurality of sensors outputting physiological data relating to the respiratory condition of the patient. The monitor includes a first transceiver configured to transmit the physiological data. The system includes an external device including a second transceiver to receive the physiological data from the second transceiver. An analytics platform is coupled to the second transceiver to analyze the physiological data received from the second transceiver to determine the occurrence of a symptom of a respiratory condition.

A further implementation of the example system is where the plurality of sensors includes a heart rate sensor and a respiratory sensor. Another implementation is where the external device is a portable computing device. Another implementation is where the analytics platform analyzes environmental data related to the patient in determining the occurrence of the symptom of the respiratory condition. Another implementation is where the analytics platform analyzes demographic data related to the patient in determining the occurrence of the symptom of the respiratory condition. Another implementation is where the plurality of sensors further includes an accelerometer. Another implementation is where the plurality of sensors further includes a pressure sensor. Another implementation is where the symptom is shortness of breath. Another implementation is where the analytics platform determines shortness of breath using a combination of: breathing effort determined from the pressure sensor and the accelerometer, and respiration rate determined from the respiratory sensor. Another implementation is where the plurality of sensors includes an audio sensor. Another implementation is where the analytics platform differentiates between a soft wheeze and other adventitious signals based on data from the audio sensor. Another implementation is where the analytics platform is executed on a remote server. Another implementation is where the analytics platform applies a model to the physiological data to determine the occurrence of a symptom of the respiratory condition. Another implementation is where the model is configured by machine learning based on collected physiological data and respiratory condition outcome data. Another implementation is where the analytics platform analyzes the physiological data to determine a risk evaluation for a respiratory event of the respiratory condition. Another implementation is where the analytics platform compares the risk evaluation with a threshold to predict the respiratory event. Another implementation is where the analytics platform initiates a corrective action in response to the predicted respiratory event. Another implementation is where the plurality of sensors includes an impedance plethysmography sensor. Another implementation is where the analytics platform is configured to determine the risk evaluation by: correlating impedance measurements from the impedance plethysmography sensor with lung volume; constructing a flow-volume curve from the lung volume; extracting one or more tidal volume parameters from the flow-volume curve; deriving features from the tidal volume parameters; and applying a model to the features to determine the risk evaluation. Another implementation is where the plurality of sensors includes an ECG sensor. Another implementation is where the analytics platform rejects noise generated by cardiac activity from the impedance measurements using the ECG sensor. Another implementation is where the plurality of sensors includes an accelerometer. Another implementation is where the analytics platform rejects movement artefacts from the impedance measurements using the accelerometer. Another implementation is where the one or more tidal volume parameters are drawn from the group consisting of: Time to Peak Expiratory Flow over Expiratory Time; Volume at Peak Expiratory Flow over Expiratory Tidal Volume; and Slope of post-peak Expiratory Flow Curve. Another implementation is where the model is configured by machine learning based on collected physiological data and respiratory condition outcome data. Another implementation is where the system includes a medication rules engine modifying a therapy plan for the respiratory condition based on the determined risk evaluation. Another implementation is where the medication rules engine is configured to adjust a dosage of a medication forming part of the therapy plan. Another implementation is where the medication rules engine is configured to adjust a type of a medication forming part of the therapy plan. Another implementation is where the analytics platform issues an alert based on the risk evaluation. Another implementation is where the system includes an alert device that receives the alert issued by the analytics platform, and alerts a person on receipt of the alert. Another implementation is where the alert device arouses the person from sleep on receipt of the alert. Another implementation is where the alert device is a wearable alert device.

Another example is a method to predict an event of a respiratory ailment in a patient. Different types of respiratory related physiological data are collected from a plurality of sensors in a monitor attached to the patient. A model to predict an event of a respiratory condition is applied. The model is based on the physiological data collected from the plurality of sensors.

A further implementation of the example method is where the plurality of sensors includes a heart rate sensor and a respiratory sensor. Another implementation is where the plurality of sensors further includes an accelerometer. Another implementation is where the plurality of sensors further includes a gyroscope. Another implementation is where the model takes into account environmental data related to the patient. Another implementation is where the model takes into account demographic data related to the patient. Another implementation is where the method includes configuring the model by machine learning based on collected physiological data and respiratory condition outcome data. Another implementation is where the method includes issuing an alert to an alert device upon prediction of the event, wherein the alert device is configured to alert a person. Another implementation is where the model includes inputs of population health factors relevant to the patient. Another implementation is where the population health factors include social determinants of health. Another implementation is where Another implementation is where the method includes inferring the social determinants of health based on a geographic location of a home of the patient. Another implementation is where the population health factors comprise data gathered from another patient in a cohort of patients that is similar to the patient. Another implementation is where the method includes initiating a corrective action in response to the predicted respiratory event. Another implementation is where the plurality of sensors includes an impedance plethysmography sensor. Another implementation is where the method further includes determining a risk evaluation by correlating impedance measurements from the impedance plethysmography sensor with lung volume. A flow-volume curve from the lung volume is constructed. One or more tidal volume parameters is extracted from the flow-volume curve. Features are derived from the tidal volume parameters. A model is applied to the features to determine the risk evaluation. Another implementation is where the plurality of sensors includes an ECG sensor. Another implementation is where the method includes rejecting noise generated by cardiac activity from the impedance measurements using the ECG sensor. Another implementation is where the plurality of sensors includes an accelerometer. Another implementation is where the method includes rejecting movement artefacts from the impedance measurements using the accelerometer. Another implementation is where the one or more tidal volume parameters are drawn from the group consisting of: Time to Peak Expiratory Flow over Expiratory Time; Volume at Peak Expiratory Flow over Expiratory Tidal Volume; and Slope of post-peak Expiratory Flow Curve.

Another disclosed example is a system to monitor a respiratory condition of a patient. The system includes a monitor attachable to the patient. The monitor has a plurality of sensors. Each of the plurality of sensors is configured to output physiological data relating to the respiratory condition of the patient. A first transceiver is configured to transmit the physiological data. An external device includes a second transceiver configured to receive the physiological data from the first transceiver. An analytics platform is coupled to the second transceiver. The analytics platform analyzes the physiological data received from the second transceiver to predict an event of the respiratory condition.

A further implementation of the example system is where the plurality of sensors includes a heart rate sensor and a respiratory sensor. Another implementation is where the plurality of sensors further includes an accelerometer.

Another implementation is where the plurality of sensors further includes an accelerometer. Another implementation is where the plurality of sensors further includes a gyroscope. Another implementation is where the model takes into account environmental data related to the patient. Another implementation is where the model takes into account demographic data related to the patient. Another implementation is where the model is configured by machine learning based on collected physiological data and respiratory condition outcome data. Another implementation is where analytics platform issues an alert to an alert device upon prediction of the event, wherein the alert device is configured to alert a person. Another implementation is where the model includes inputs of population health factors relevant to the patient. Another implementation is where the population health factors include social determinants of health. Another implementation is where the analytics platform infers the social determinants of health based on a geographic location of a home of the patient. Another implementation is where the population health factors comprise data gathered from another patient in a cohort of patients that is similar to the patient. Another implementation is where analytics platform initiates a corrective action in response to the predicted respiratory event. Another implementation is where the plurality of sensors includes an impedance plethysmography sensor. Another implementation is where the analytics platform is configured to determine a risk evaluation by correlating impedance measurements from the impedance plethysmography sensor with lung volume. A flow-volume curve from the lung volume is constructed. One or more tidal volume parameters is extracted from the flow-volume curve. Features are derived from the tidal volume parameters. A model is applied to the features to determine the risk evaluation. Another implementation is where the plurality of sensors includes an ECG sensor. Another implementation is where the analytics platform rejects noise generated by cardiac activity from the impedance measurements using the ECG sensor. Another implementation is where the plurality of sensors includes an accelerometer. Another implementation is where analytics platform rejects movement artefacts from the impedance measurements using the accelerometer. Another implementation is where the one or more tidal volume parameters are drawn from the group consisting of: Time to Peak Expiratory Flow over Expiratory Time; Volume at Peak Expiratory Flow over Expiratory Tidal Volume; and Slope of post-peak Expiratory Flow Curve.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a block diagram of a continuous monitoring system to monitor respiratory conditions, disorders and ailments and determine corresponding symptoms, including an example continuous monitoring device on a patient;

FIG. 2 is a block diagram of the electronic components of the continuous monitoring device and other elements of the system in FIG. 1;

FIG. 3 is a flow diagram of an example machine learning process to train a predictive model for an example respiratory ailment such as asthma;

FIG. 4 is a flow diagram of a routine to gather and process the data from the continuous monitoring device in FIG. 1;

FIGS. 5A to 5B are graphs of example collected signal data for the output of different sensors on the continuous monitoring device in FIG. 1;

FIG. 5C is a graph of example collected signal data containing movement artifacts from the data analyzed from the continuous monitoring device of FIG. 1;

FIG. 5D is a graph illustrating rejection of cardiogenic noise from the data analyzed from the continuous monitoring device of FIG. 1;

FIG. 6 is a block diagram of the data flow in a system that collects data from the continuous monitoring device in FIG. 1;

FIG. 7 is a block diagram of a health care system that incorporates and supports the continuous monitoring system in FIG. 1;

FIG. 8A is a perspective view of an example continuous monitoring device for use with the system in FIG. 1;

FIG. 8B is a circuit layout of the example monitoring device in FIG. 8A;

FIG. 8C is a top perspective view of the internal components of the example monitoring device in FIG. 8A;

FIG. 8D is a bottom perspective view of the internal components of the example monitoring device in FIG. 8A;

FIG. 9 is a block diagram of the components of the example monitoring device in FIG. 8A;

FIG. 10A is a perspective view of an example adhesive accessory for applying the example monitoring device in FIG. 8A prior to application;

FIG. 10B shows successive steps in applying the adhesives in the adhesive accessory of FIG. 10A to the monitoring device in FIG. 8A before application to the skin of a patient;

FIG. 11 is a process flow diagram showing one example of collection of data from a monitoring device and predictive analysis thereon; and

FIG. 12 shows two graphs illustrating a flow-volume curve and the tidal volume parameters that may be extracted from such a curve.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a continuous monitoring system for monitoring respiratory conditions, disorders or ailments, such as asthma in a patient. The system has a continuous monitor that is attached to the patient. The monitor has sensors that take multiple physiological readings from the patient. The data from the readings may be transmitted to an external device. The system includes a machine learning engine that allows analysis and determination of data that are indicative of symptoms of the respiratory conditions, disorders or ailments. The system may use data to predict respiratory events such as asthma attacks. The patient or family member of the patient may be alerted so as to take preventive measures.

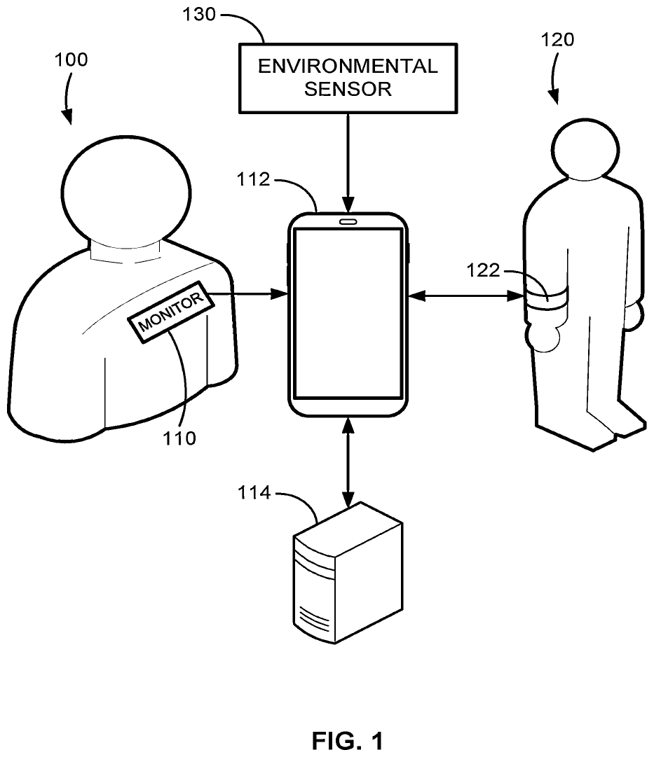

FIG. 1 shows a patient 100 that has a continuous respiratory monitoring device 110 (monitor) applied to the chest. As will be explained the monitor 110 can be applied anywhere on the body of the patient 100 that allows sensing of relevant physiological signals from the patient 100. In this example, the respiratory monitor 110 includes a transmitter for data transmission, a sensor or sensors for sensing respiratory-related signals, and an adhesive for attachment to the patient 100. The monitor 110 may be replaced on a periodic basis, but is compact and may stay on the patient 100 for the monitoring period. The monitor 110 may also be reusable. The monitor 110 thus may obtain continuous data from the patient to monitor respiratory conditions, disorders, or ailments. The data sensed by the monitor 110 may be transmitted to a remote external portable device 112 such as a smart phone. The portable device 112 may be in communication with an external data server 114 through a network such as the Internet or the Cloud. The data server 114 may execute applications for data analysis and machine learning in relation to determining symptoms of respiratory conditions, disorders, or ailments, as well as predicting respiratory events as will be explained below.

The monitor 110 generally will include a flat protective enclosure that encloses electronic components such as the power source, transceiver, memory, controller, sensor interfaces and sensor electronics. In this example, the enclosure is fabricated from a flexible material such as silicone in order to flex with the skin of a user. A sensor interface area or areas may be placed in contact with the skin of the patient. Such sensor contact areas may include ECG electrode pads, impedance electrode pads, acoustic pads, or PPG sources and detectors. Certain electrodes may be used by multiple sensors. The monitor 110 may have different wearable form factors such as a patch, a wristband, a necklace or a vest.

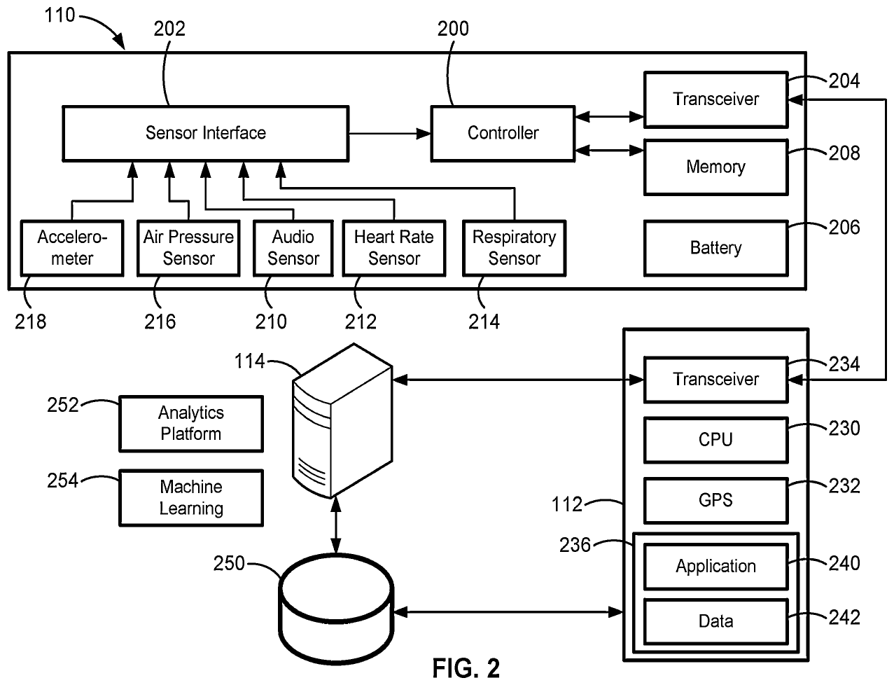

FIG. 2 is a block diagram of the electronic components of the monitor 110, the portable device 112, and the external server 114. The monitor 110 includes a controller 200, a sensor interface 202, a transceiver 204, a memory 206, and a battery 208. The sensor interface 202 is in communication with an audio sensor 210, a heart rate sensor 212, a respiratory sensor 214, a contact pressure sensor (strain gauge) 216, and an optional accelerometer 218.

The transceiver 204 allows exchange of data between the monitor 110 and the remote external portable device 112 in FIG. 1. The transceiver 204 in this example is a wireless link that may incorporate any suitable wireless connection technology known in the art, including but not limited to Wi-Fi (IEEE 802.11), Bluetooth, other radio frequencies, Infra-Red (IR), GSM, CDMA, GPRS, 3G, 4G, W-CDMA, EDGE or DCDMA200 and similar technologies.

The memory 206 may store computer modules or other software to configure the controller 200 to implement the functions of monitor 110 described herein. Additionally, the memory 206 may store data collected by the various sensors associated with monitor 110. This data may be continually transmitted to associated devices for long term storage or stored on memory 206 until downloaded by connecting another device to the monitor 110.

In this example the audio sensor 210 detects sounds from the lungs. Such sounds may be indicative of symptoms of, and predictive of respiratory events occurring in, respiratory conditions, disorders, or ailments. For example, wheezing or coughing sounds may be predictive of a future asthma attack. Such predictions may also be made from the audio data in combination with other data such as heart rate. In this example, the heart rate sensor 212 is a two lead electrocardiogram (ECG) sensor. In this example, the respiratory sensor 214 is an impedance plethysmography (IPG) sensor having two voltage leads and two current leads. The example monitor 110 includes an optional pressure sensor 216 and an optional accelerometer 218. In this example, data from the different sensors 210, 212, 214, 216 and 218 may be analyzed for determining symptoms of respiratory conditions, disorders, or ailments and predicting respiratory events. For example, sensor data from the pressure sensor 216 and the accelerometer 218 may be used to determine tidal volume of the lung. Pressure data from the pressure sensor 216 may be used to measure breathing effort. The tidal volume and breathing effort taken together may be predictive of a respiratory event such as an asthma attack.

Other sensors may be part of the monitor 110. Such sensors may include doppler radar motion sensors, thermometers, scales, or photoplethysmography (PPG) sensors, each of which is configured to provide additional physiological data (biomotion, temperature, weight, and oxygen saturation respectively) measured from the patient 100. The additional sensors may be used to provide additional types of data, which may be analyzed, either alone or with other types of data, to determine symptoms of respiratory conditions, disorders, or ailments and predict respiratory events. The additional sensors or the sensors 210, 212 and 214 may also be used for other purposes such as heart rate variability (HRV) monitoring. There may also be data obtained from external sensors such as an environmental sensor 130. Such an environmental sensor 130 may transmit data such as external temperature, humidity, or pollen count to the portable device 112 or the server 114 to assist in predictive analysis.

The remote external portable device 112 may be a portable computing device such as a smart phone or a tablet that may execute applications to collect, analyze and display data from the monitor 110. The remote external portable device 112 may include a CPU 230, a GPS receiver 232, a transceiver 234, and a memory 236. The memory 236 may include an application 240 for collecting and analyzing data. The memory 236 also stores the collected data 242 received from the monitor 110. Additional data such as patient specific data or environmental data that may be used in determining symptoms of respiratory conditions, disorders, or ailments and predicting respiratory events may also be stored in the memory 236. The additional data may also be analyzed and compiled by the application 240. The remote external portable device 112 may have access to a database 250 that includes “big data” from other monitors and corresponding patients. The patient application 240 may be operable to provide the patient or the family of the patient actionable insights and recommendations for controlling respiratory events such as anticipation of asthma attacks.

The server 114 may also have access to the database 250. The server 114 may run one or more analysis algorithms as part of an analytics platform 252 that are configured by machine learning to analyze the data received from the external portable device 112 and monitor the respiratory condition of the patient. The server 114 may also execute a machine learning module 254 that configures the analysis algorithm(s) to both determine symptoms and predict respiratory events from the collected data.

The algorithm(s) for monitoring respiratory conditions, disorders, or ailments may analyze the data from the sensors 210, 212, and 214 or data that is produced as a result of refining or combining the data from the sensors 210, 212, and 214. As explained above, the algorithm determines symptoms of respiratory conditions, disorders, or ailments. The algorithm may be performed by the patient application 240 or may be performed by the analytics platform 252. The results of the analysis may be made available directly to the patient or the family of the patient via an interface generated by the application 240 on the portable device 112. The application 240 may also provide suggested courses of corrective action such as take medications, call a health professional, or cease exertion, to the patient or the family of the patient. Of course, these determinations may also be made available to the server 114.

As explained below, a predictive algorithm for predicting respiratory events may also be executed by the server 114. Such an algorithm may provide additional analysis to that performed by the application 240 on the portable device 112. The predictive analysis may be made available to other actors such as health care providers based on the patient or the family of patient providing permission. The predictive analysis may be used for different purposes such as formulating an action for the patient. Such an action may comprise recommending medication, increasing or decreasing the frequency of medication, or advising to change activity based on the severity of the respiratory event predicted by the algorithm.

As shown in FIG. 1, a family member 120 such as a parent may operate the portable device 112 and receive information and recommendations in relation to the patient 100. For example, the family member 120 may receive alerts in relation to the condition of the patient 100. Alternatively, the family member 120 may have a wearable networked alert device 122 such as a smart watch, a bracelet, a necklace, or a headband that receives alerts from either the portable device 112 or the server 114. For example, an alert may be issued to the family member 120 when a respiratory event is predicted or detected by the portable device 112 or determined by the algorithm executed by the server 114. The alert may be sent to the portable device 112 associated with the family member 120. Alternatively, or in addition, the alert may be sent to the wearable networked alert device 122 to better ensure the family member 120 receives the alert. This better ensures that the family member 120 is notified of the status of the patient 100, especially at night, via an application running on a wearable networked alert device 122. The notification or alert may also be received by a smart-home or Internet of Things (IoT) networked appliance (e.g. light, alarm clock, baby monitor, CPAP device, smart mattress) that is in proximity to the family member 120 and is configured to arouse the family member by visual, auditory, tactile or other like means.

Thus, the algorithms running on either the external portable device 112 or the server 114 may determine symptoms of respiratory conditions, disorders, or ailments and may predict respiratory events. For example, the algorithm may determine the symptom of shortness of breath using a combination of breathing effort and respiration rate. Breathing effort may be determined from the readings of the pressure sensor 216 and the intensity of chest movement from the accelerometer 218, or the respiratory sensor 214. Another example of a symptom is determining changes in the inspiration to expiration ratio, which can be an early indicator of a respiratory event such as an asthma attack. Leading up to an asthma attack the ratio between inspiration to expiration decreases, meaning the inspiration shortens and patients tend to expire for a longer period of time to get more air out of inflamed lungs. The inspiration to expiration ratio may be measured using the audio sensor 210 and the respiratory sensor 214.

The algorithms may also determine change of lung volume to predict a respiratory event. The change of lung volume may be related to audio signals, or heart rate data, or respiratory data. Lung volume may be measured without the audio sensor 210 using the heart rate and respiratory data alone. Changes in lung volume may be correlated with changes in impedance determined by the IPG sensor 214.

An impedance signal from the IPG sensor 214 may be used to determine belly breathing. The belly breathing indicates lung airways narrowing and de-synchronized patterns compared to upper chest movement. Thus, belly breathing is an indicator of a patient struggling to breathe due to inflamed or congested airways or lungs. The algorithm may also determine heart rate variability based on data from the heart rate sensor 212. The heart rate may be correlated as a measure of the autonomous nervous system. Heart rate variability is a measure of the sympathetic and parasympathetic nervous system which can be used to measure the level of anxiety and stress. The heart rate can also be used to detect medication intake as Bronchodilators often result in high heart rate.

The algorithms may also determine night-time awakening and other indicators of sleep quality using a combination of movement, heart rate and breathing. The algorithms may correlate readings from the accelerometer 218 indicating movement, respiration rate data from the respiratory sensor 214, and variability in heart rate determined from the heart rate sensor 212.

The algorithms may also analyze the audio signal output from the audio sensor 210 to differentiate between a soft wheeze and other adventitious signals. Thus, the algorithms have the ability to determine intensity and timing (inspiration or expiration) of a wheeze sound. The intensity and timing of the wheeze sound may be a symptom of respiratory conditions, disorders, or ailments. The changes in intensity and timing of such sounds may also be used to predict a respiratory event.

The algorithms may combine multiple sensor signals to pick up “silent chest,” an indicator of severe asthma. The silent chest condition is one where the audio sensor 210 does not pick up any signal but other vital signs like heart rate and respiration rate from the sensors 212 and 214 will be very high with high variability. It is the combination of all these signals that enable the algorithm to determine or predict the occurrence of a respiratory event such as a severe asthma attack. Further, using the multiple sensors, the algorithm may determine symptoms of a respiratory condition across the full spectrum of asthma from mild asthma all the way to severe asthma based on a multi-sensor approach from the audio, heart rate and respiratory data collected from the sensors 210, 212, and 214.

The algorithms and monitor 110 may be combined with treatment devices such as inhalers. For example, the algorithms may have the ability to detect if inhaler technique is proper to ensure medication was taken correctly. For example, the algorithm may take an input from an adherence monitor as described in U.S. Pat. No. 9,550,031, to Reciprocal Labs Corp in combination with an inhaler to allow comparing the timing of inhaler click with the expiration/inhalation from the sensors on the monitor 110.

The data outputs of the monitor 110 may also be combined with other sensor inputs external to the monitor 110 or other data collected from other sources. For example, the algorithms may consider alerts of exposure to environmental triggers based on location information obtained from the GPS receiver 232 or a built-in GPS sensor on the portable device 112 correlated to data relating to local weather conditions.

The combination of determined symptoms may generate an individualized risk evaluation such as the probability of a respiratory event such as an asthma attack. Such a risk evaluation may also take into account manually entered data such as patient history and clinical recommendations. The risk evaluation can then be translated into a set of ranges that may be used to output the risk evaluation to the patient, the family of the patient or a health care professional. For example, the resulting ranges may be displayed on a user interface on the portable device 112.

This data collected from the monitor 110 and other monitors from similar patients may serve as a predictive indicator for how similar patients may respond to similar environments, therapy plans, and what may trigger respiratory events in similar patients. The analytics platform 252 uses a model to predict respiratory events based on different data inputs. The model may be a known model or a model configured by the machine learning module 254. Predictive data may be used to allow a system to issue alerts for impending respiratory events such as asthma attacks to patients or family members of patients. The predictive data may be provided to health care providers to evaluate and modify a therapy plan or recommend preventive medication for such respiratory events.

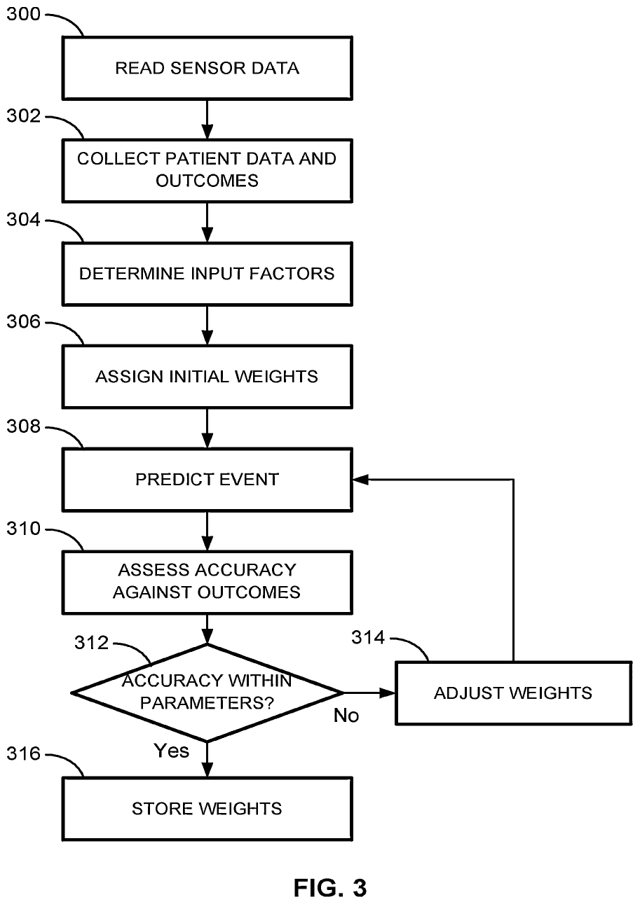

FIG. 3 is an example routine to train a respiratory condition model, e.g. a neural network, to predict respiratory events. The example routine may be part of the machine learning module 254 executed by the server 114 in FIG. 1. In this example, the routine in FIG. 3 is unsupervised learning based on data from sensors and patient specific data including demographic data and outcome data based on the patients' respiratory conditions. The routine collects sensor data from each of the sensors such as the sensors 210, 212, and 214 of the monitor 110 for monitors attached to numerous patients as inputs (300). The routine then collects corresponding patient specific data including demographic data as additional inputs and outcome data such as respiratory events of the corresponding patients as outputs (302). The routine determines a potential set of input factors that are predictive of respiratory events based on the collected data (304). The routine then assigns weights to the input factors (306). The routine then attempts to predict the output respiratory events based on the weighted input factors (308). The routine then assesses the accuracy of the predictions (310). If the accuracy does not meet a desired level (“No” at 312), the routine adjusts the weights (314) and loops back to the prediction step (308). If the accuracy meets the desired level (“Yes” at 312), the routine stores the weights (316) and the resulting model may be deployed to provide analysis based on the input sensor signals from monitors such as the monitor 110.

Thus, the neural network in this example, may be provided with respiratory related data collected from each of the patients by monitors such as the monitor 110. In addition, patient specific data may be collected from inquiries made on a patient computing device such as the portable device 112 or imported from electronic medical record databases. Further information may be stored based on the data collected from monitors such as the monitor 110. Additionally, patient specific data on other patients such as demographic information, medical histories, and genetic makeup, may be provided to the neural network.

The sensor information may be processed by a neural network that may determine patterns based on the received sensor data. Additionally, other factors may be provided to the model. The neural network may also determine patterns based on data relating to patient demographics relating to respiratory conditions, ailments, or disorders, such as geographic location, weather, medical history, and environmental factors. Additionally, the neural network may be able to determine patterns that indicate the effect of medication and treatment on the frequencies and severities of respiratory events.

Once the neural network has established patterns and created a model, the data collected by the monitor 110, and other information such as location data and patient specific data from the patient may be processed by the neural network. Accordingly, the neural network may provide a model that determines symptoms of respiratory conditions, ailments, or disorders and predicts respiratory events based on multiple types of data. This output data may then be utilized by health care professionals, the family of the patient or the patient to guide preventive measures or treatments. For example, applications may use the output data to prepare reports that indicate high risk factors for respiratory events to a specific patient. Such reports may be sent to the external portable device 112 or communicated to the patient in another way.

For example, the neural network may determine that there is a high likelihood that certain environments or locations may worsen respiratory conditions, ailments, or disorders or cause respiratory events. For example, a patient may be traveling to a new location. Once the patient arrives at the destination, the associated external portable device 112 may send location data to the server 114 for input to the neural network. Accordingly, the neural network may then determine that a respiratory event is likely because similar patients experienced such events in the area or under similar conditions. The model may be continuously updated by new input data from monitors such as the monitor 110 and other sources, as well as resulting respiratory symptoms. Thus, the model may become more accurate with greater use by the analytics platform 252.

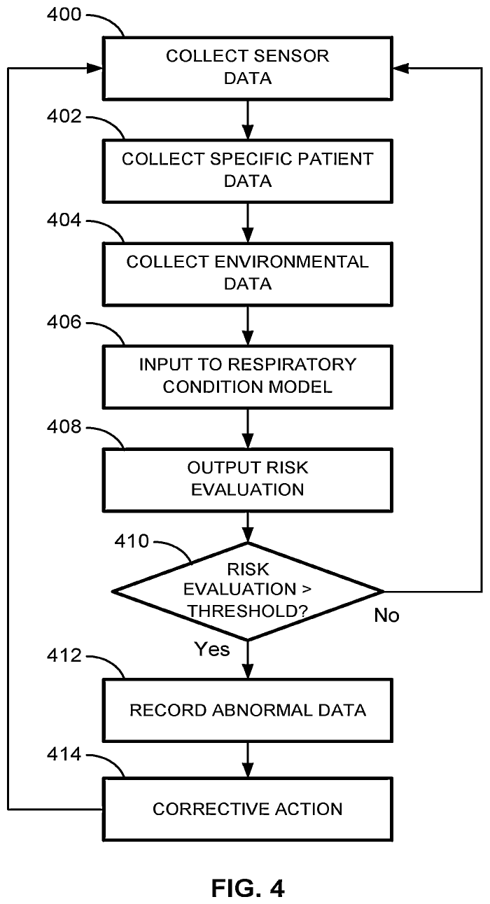

FIG. 4 is an example routine for the collection and analysis of data in the system shown in FIG. 2. The routine first collects sensor data from the monitor 110 (400). The collected sensor data may be in summary form for the audio signal of lung sounds over time, heartbeats over time or respiration rate over time. Additional data may be derived from one or more of the sensor outputs such as actimetry, impedance plethysmography, or temperature. The routine collects patient specific data from a medical record database (402). The routine then collects relevant environmental data such as humidity, altitude, pollen count, etc. (404). Such environmental data may be obtained from databases or sensors on either the monitor 110 or the portable device 112.

The relevant data is then input into the respiratory condition model (406). The model evaluates the relevant data according to weightings determined by the machine learning process in FIG. 3. The model outputs a risk evaluation for respiratory events (408). The routine then determines whether the risk evaluation exceeds a predetermined threshold (410). If the risk evaluation does not exceed the predetermined threshold (“No” at 410), the routine continues to collect data (400).

If the risk evaluation exceeds a predetermined threshold (“Yes” at 410), that is, a respiratory event is predicted, the routine will store the abnormal data (412) whose analysis resulted in the predicted event. The abnormal data may be forwarded to a health care professional or other applications for further analysis or action. The abnormal data may also be added to a patient health record. The routine will then initiate corrective action (414). Corrective action may include alerts to the patient or the family of the patient or health care professionals.

The flow diagrams in FIGS. 3-4 are representative of example machine readable instructions for collecting and analyzing data to predict respiratory events. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithms are described with reference to the flowcharts illustrated in FIGS. 3-4, persons of ordinary skill in the art will readily appreciate that many other methods of implementing the example machine readable instructions may alternatively be used. For example, the order of execution of the blocks may be changed, and/or some of the blocks described may be changed, eliminated, or combined.

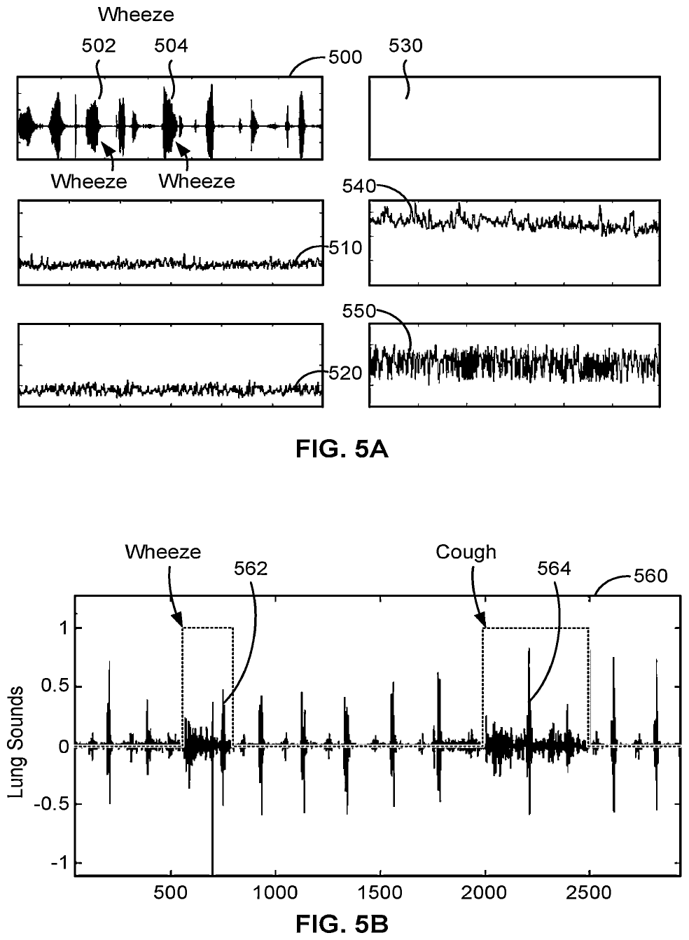

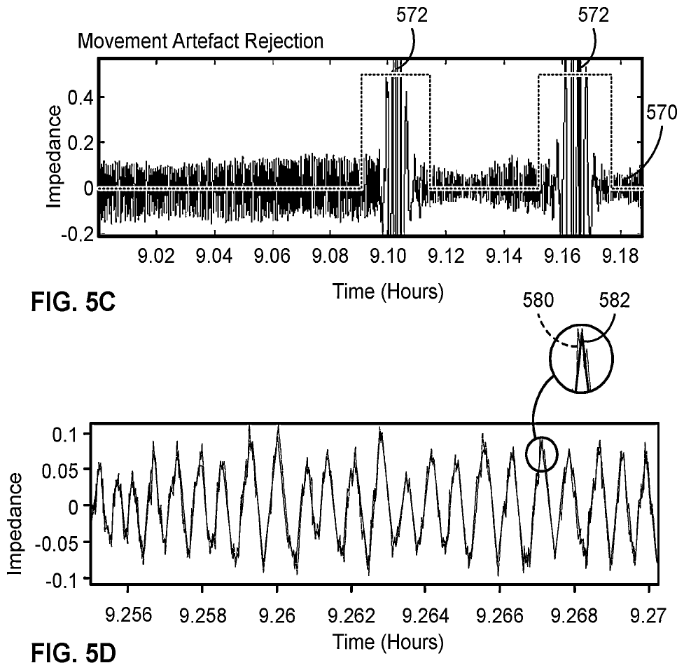

FIG. 5A shows example waveforms that are based on the output of different sensors 210, 212 and 214 from the monitor 110 in FIG. 2 that may be used by the algorithm to predict a respiratory event such as the onset of a severe asthma attack. The data shown in the waveforms in FIG. 5A are an example of predicting respiratory events based on multiple different sensor data. FIG. 5A shows an early stage lung audio waveform 500, an early stage heartbeat waveform 510, and an early stage respiratory waveform 520. The early stage output waveforms 500, 510 and 520 may be used in combination by the routine described above to determine symptoms of respiratory conditions, ailments, or disorders. The output waveform data for the output of sensors 210, 212 and 214 is stored in the monitor 110 for retrieval by an external client device such as the portable device 112 that then transmits the data to a server executing the analysis routine such as the server 114.

In this example, the early stage lung audio waveform 500 shows peaks 502 and 504 that indicate a wheezing sound from the lungs. A late stage lung audio waveform 530 shows a lack of any audio signal demonstrating the potential of “silent chest” indicating a severe asthma attack. The early stage heartbeat waveform 510 shows relatively short consistent peaks. In contrast, a late stage heartbeat waveform 540 shows higher magnitude beats and more variation in the heartbeat indicating higher sympathetic nervous system activity, which is an indicator of stress due to severe asthma attack. The early stage respiratory waveform 520 shows relatively low magnitude variation between peaks. In contrast, a late stage respiratory waveform 550 shows high variation between greater peaks indicating a patient struggling to breathe due to narrow lung airways. The combination of the data from the late stage waveforms 530, 540, and 550 may allow the algorithm to more accurately predict the onset of an asthma attack. The data may also allow a determination of the severity of the attack, allowing a more heightened response.

FIG. 5B is an example audio waveform 560. As explained above, the learning algorithm may correlate different signals to predict respiratory events. For example, the learning algorithm may determine that specific signatures 562 and 564 represent a wheezing sound and a coughing sound respectively. The signatures 562 and 564 may be correlated to a symptom of a respiratory disorder. Thus, the algorithm may also predict respiratory events based on a single type of data alone or a single type of data combined with other different types of data.

Other analysis may be performed to determine respiration rate and lung volume. For example, lung volume may be correlated with impedance measurements. As described in more detail below, parameters may be determined from a flow-volume curve that is constructed by plotting respiratory flow rate against lung volume.

FIG. 5C is a graph of example collected signal data containing movement artifacts from the data analyzed from the continuous monitoring device 110. In this example, impedance data 570 is taken from the respiratory sensor 214 in FIG. 2. The impedance data 570 may be processed to reject movement artifacts generated by bodily movement as detected by an accelerometer such as the accelerometer 218. Certain peaks 572 indicate bodily movement that may then be ignored in the analysis of the impedance data 570.

FIG. 5D is a graph illustrating rejection of cardiogenic noise from the data analyzed from the continuous monitoring device 110. In this example, impedance data taken from the sensor 214 in FIG. 2 is plotted as a trace 580. The impedance data may be processed to reject noise generated by cardiac activity as detected by an ECG sensor such as the heart rate sensor 212. Thus, certain peaks in the impedance waveform 580 may be filtered to a modified trace 582 to minimize cardiogenic noise as detected by the heart rate sensor 212. In one implementation, R-peaks from the ECG sensor may be used as a trigger.

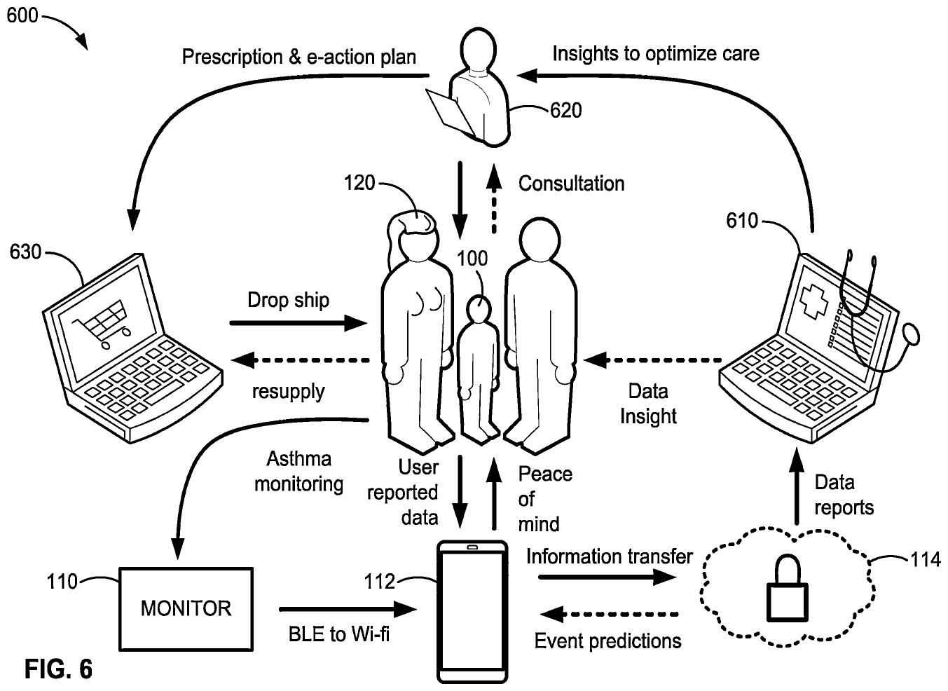

FIG. 6 is a block diagram of the data flow in a system 600 for monitoring respiratory conditions, ailments, or disorders in patients such as the patient 100. As shown in FIG. 6, data from the monitor 110 is collected by the application executed on the portable device 112. Additional patient specific, medical or demographic information may be manually entered by the patient or a family member 120 of the patient to the portable device 112. Such information may also be obtained from medical record databases. The portable device 112 may provide information based on the collected data to the patient or their family on different interfaces as explained above.

The portable device 112 may directly send collected data from the monitor 110 and/or send analyzed data to an analytics platform executed on a server such as the server 114 via a network such as the Internet or the Cloud. As explained above, the analytics platform may provide symptoms of respiratory conditions, ailments, or disorders and predictive analytics data as to respiratory events. The output may be made in the form of data reports that may be transmitted to a health care provider system 610. The health care provider system 610 may provide additional insights to either the patient or the family of the patient directly or to a health care professional 620. In this example, the health care professional 620 may prescribe preventive medication from a supply system 630 that may ship the preventive medication such as anti-inflammatories, as well as treatment devices, such as inhalers, to the patient 100.

Several interfaces may be displayed on the patient device 112. The interfaces may display the determined symptoms and risk evaluations of respiratory events. For example, an interface may display a traffic light system where green indicates normal risk, orange indicates a heightened risk, and red indicates a high risk based on the collected data. Thus, an example interface may provide information in understandable fashion, giving peace of mind to the family of the patient 100. Other interfaces may allow a patient or the family of a patient to contact a health care professional or send analyzed data to the health care professional.

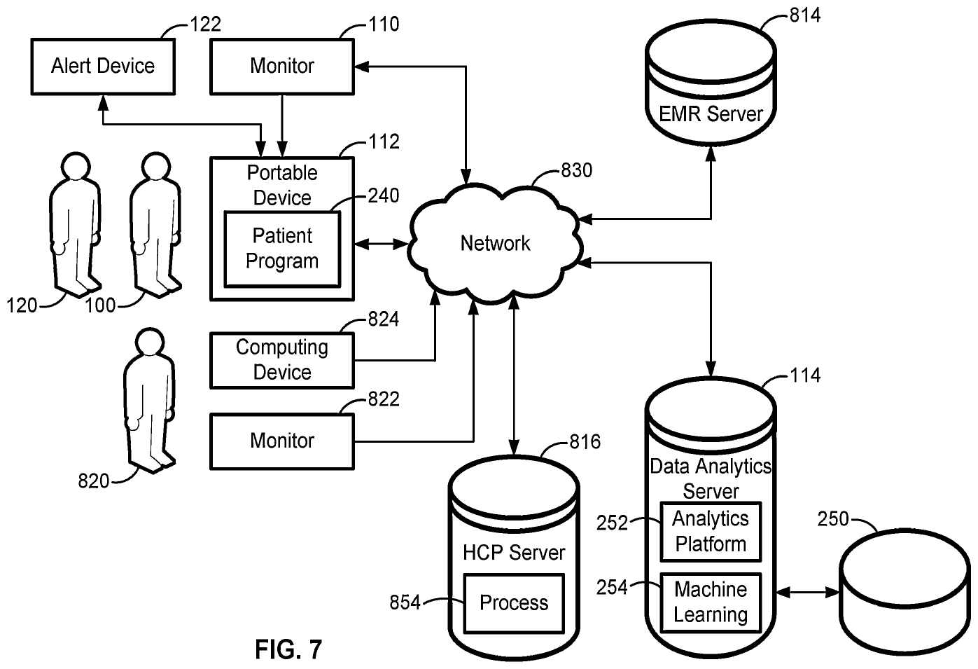

FIG. 7 is a block diagram of an example health care system 800 for obtaining data from patients having an attached monitor such as the monitor 110 in FIG. 1. The health care system 800 includes the server 114, an electronic medical records (EMR) server 814, a health or home care provider (HCP) server 816, the external portable device 112, and the monitor 110 from FIG. 1. The portable device 112 and the monitor 110 are co-located with the patient 100 in this example. In the system 800, these entities are all connected to, and configured to communicate with each other over, a wide area network 830, such as the Internet. The connections to the wide area network 830 may be wired or wireless. The EMR server 814, the HCP server 816, and the data server 114 may all be implemented on distinct computing devices at separate locations, or any sub-combination of two or more of those entities may be co-implemented on the same computing device.

The portable device 112 may be a personal computer, smart phone, tablet computer, or other device. The portable device 112 is configured to intermediate between the patient 100 and the remotely located entities of the system 800 over the wide area network 830. In the implementation of FIG. 7, this intermediation is accomplished by the software application program or application 240 that runs on the portable device 112. The patient program 240 may be a dedicated application referred to as a “patient app” or a web browser that interacts with a website provided by the health or home care provider. Alternatively, the monitor 110 may communicate with the portable device 112 via a local wired or wireless network (not shown) based on a protocol such as Bluetooth. The system 800 may include other patients 820 that provide data through respective monitors 822 and portable devices 824. All the patients in the system 800 may be managed by the data server 114.

As explained above, the data from the monitor 110 and/or portable device 112 may be collected to predict respiratory events via the analytics platform 252 on the data server 114. As previously explained, a family member such as a parent 120 may receive alerts about the patient 100 via a wearable networked alert device 122 similar to the portable device 112. Alternatively, the family member 120 may wear the alert device 122 to receive alerts from the portable device 112 or the data server 114. The analytics platform 252 may provide analysis of the collected data using the routine in FIG. 4 to determine symptoms and predict respiratory events. Additional data from the monitor 110 may be collected for other purposes such as tracking the effectiveness of preventive measures or treatments, tracking sleep quality, anxiety and stress. The combination of physiological signals derived from multiple sensors on the monitor 110 such as respiration rate, heart rate, and body position can be used to detect sleep/wake and classify sleep stages. These physiological signals can further be used to detect apnea and hypopnea which can help in the diagnosis of sleep disordered breathing. The ECG signal from an ECG sensor such as the heart rate sensor 212 may further be used to monitor sympathetic and parasympathetic nervous system response through frequency analysis of heart rate variability (HRV). HRV is a promising biomarker of mental health resilience and is an index of flexibility and ability to adapt to stress.

Such data may be transmitted by either the monitor 110 or the portable device 112 to the data server 114. The data server 114 may also execute the machine learning module 254 to further refine a model for correlating data with respiratory events to increase the accuracy of the predictions of the analytics platform 252.

In this example, the monitor 110 is configured to transmit the physiological data from continuous monitoring of different respiratory related sensors to the portable device 112 via a wireless protocol, which receives the data as part of the patient program 240. The portable device 112 then transmits the data to the data server 114 according to pull or push model. The data server 114 may receive the physiological data from the portable device 112 according to a “pull” model whereby the portable device 112 transmits the physiological data in response to a query from the data server 114. Alternatively, the data server 114 may receive the physiological data according to a “push” model whereby the portable device 112 transmits the physiological data to the data server 114 as soon as it is available after a pre-determined period of time. The data server 114 may access databases such as the database 250 to store collected and analyzed data.

Data received from the portable device 112 is stored and indexed by the data server 114 so as to be uniquely associated with the patient 100 and therefore distinguishable from physiological data collected from any other patients 820 in the system 800. The data server 114 may be configured to calculate summary data from the data received from the monitor 110. The data server 114 may also be configured to receive data from the portable device 112 including data entered by the patient 100 or the family of the patient, behavioral data about the patient, or summary data.

The EMR server 814 contains electronic medical records (EMRs), both specific to the patient 100 and generic to a larger population of patients with similar disorders to the patient 100. An EMR, sometimes referred to as an electronic health record (EHR), typically contains a medical history of a patient including previous conditions, treatments, co-morbidities, and current status. The EMR server 814 may be located, for example, at a hospital where the patient 100 has previously received treatment. The EMR server 814 is configured to transmit EMR data to the data server 114, possibly in response to a query received from the data server 114.

In this example, the HCP server 816 is associated with the health/home care provider (which may be an individual health care professional or an organization) that is responsible for the treatment and care of the patient 100 such as for respiratory therapy. An HCP may also be referred to as a DME or HME (domestic/home medical equipment provider). The HCP server 816 may host a process 854 that is described in more detail below. One function of the HCP server process 854 is to transmit data relating to the patient 100 to the data server 114, possibly in response to a query received from the data server 114.

In some implementations, the data server 114 is configured to communicate with the HCP server 816 to trigger notifications or action recommendations to an agent of the HCP such as a nurse, or to support reporting of various kinds. Details of actions carried out are stored by the data server 114 as part of the engagement data. The HCP server 816 hosts an HCP server process 854 that communicates with the analytics platform 252 and the patient program 240.

For example, the HCP server process 854 may include the ability to monitor the patient in relation to use of treatment medication or devices such as an inhaler with compliance rules that specify the required inhaler usage over a compliance period, such as 30 days, in terms of a minimum number of doses, such as four times, for some minimum number of days, e.g. 21, within the compliance period. The summary data post-processing may determine whether the most recent time period is a compliant session by comparing the usage data with the minimum number from the compliance rule. The results of such post-processing are referred to as “compliance data.” Such compliance data may be used by a health care provider to tailor therapy that may include the inhaler and other mechanisms. Other actors such as payors may use the compliance data to determine whether reimbursement may be made to a patient. The HCP server process 854 may have other health care functions such as determining overall use of drugs based on collection of data from numerous patients. For payors, compliance data may help phenotype non-compliant patients and recommend they be put on alternative treatments such as biologics.

As may be appreciated, data in the data server 114, EMR server 814 and HCP server 816 is generally confidential data in relation to the patient 100. Typically, the patient 100 or family member 120 of the patient must provide permission to send the confidential data to another party. Such permissions may be required to transfer data between the servers 114, 814 and 816 if such servers are operated by different entities.

The continuous monitoring in the system in FIG. 7 may be used for a variety of respiratory disorders such as asthma, COPD, cystic fibrosis, and bronchiectasis. However, it is to be understood and appreciated that the principles described above are not to be limited to such use.

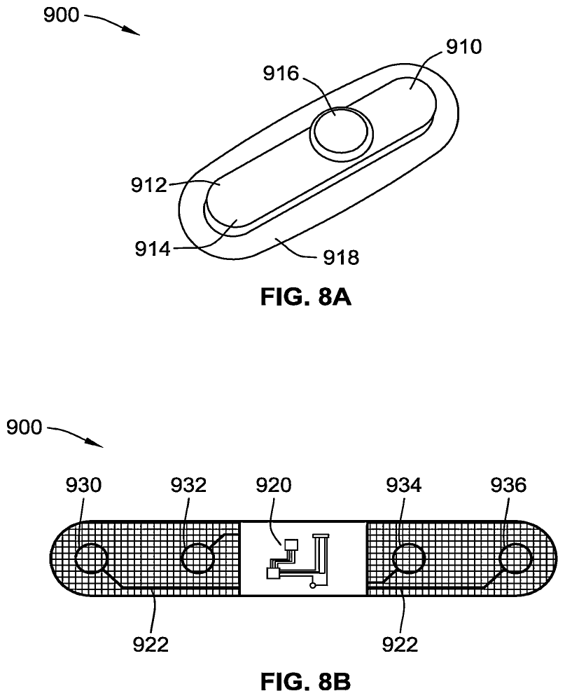

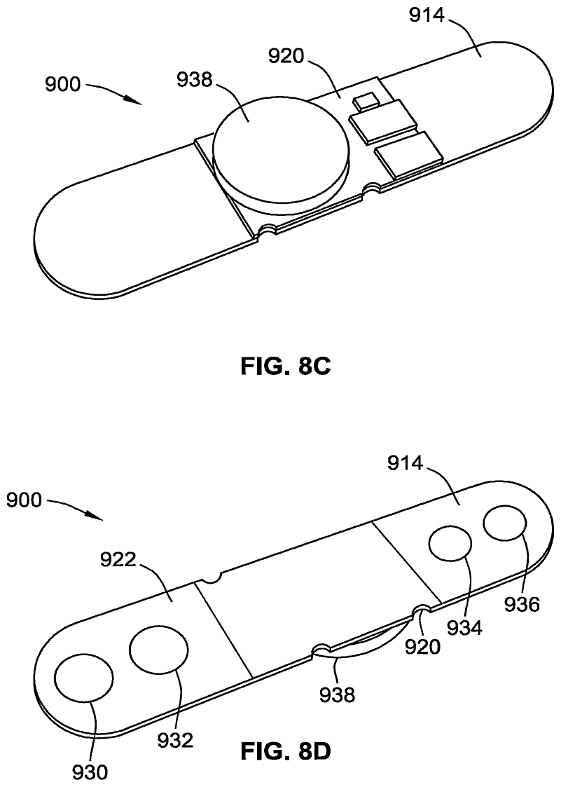

FIG. 8A is a perspective view of an example patch type monitor 900 that may be used for the monitor 110 shown in FIG. 1. FIG. 8B is a circuit layout of the example monitoring device 900. FIG. 8C is a top perspective view of the internal components of the example monitoring device 900. FIG. 8D is a bottom perspective view of the internal components of the example monitoring device 900. The monitoring device 900 has similar functions to the monitor 110 insofar as it collects time-dependent physiological data signals from a patient and sends the data to a portable device such as the portable device 112 in FIG. 1. Thus, the example monitor 900 collects cardio-respiratory signals from the chest of a patient and stores them in an on-board memory from which the stored data can be downloaded to a smart phone/tablet via Bluetooth.

The monitoring device 900 includes an enclosure 910 that has a top surface 912 and a bottom surface 914. In this example, the enclosure 910 is a silicone shell casing, but other suitable flexible compliant materials that allow flexing to conform with skin movements may be used. In this example, the enclosure 910 has a length of 90 mm and a width of 20 mm, but other suitable dimensions and shapes may be used for the enclosure. As will be explained the bottom surface 914 is a contact surface that is attached to a layer 918 that has adhesives that are applied to the bottom surface 914. The layer 918 also has adhesives on its underside that are configured to attach the layer 918 to the skin of the patient. As will be explained below, the layer 918 is part of an adhesive accessory that may be used to adhere the monitor enclosure 910 to the chest of a patient in one implementation of the present technology. The monitor 900 is intended to be attached horizontally on the upper medial part of the chest of the patient, but other orientations such as at 45 degrees to the horizontal, and other locations such as on the upper left or right chest or on the ribs below the right or left armpit are contemplated. The top surface 912 includes a cylindrical battery housing 916.

FIG. 8B shows a circuit board 920 that is housed in the enclosure 910. The circuit board 920 includes all of the sensors, the memory, transceiver, microprocessor, signal processor, and other electronic components as will be explained herein. Traces 922 are attached to circular electrode pads 930, 932, 934, and 936 that are formed in the bottom surface 914 of the enclosure 910. In one implementation, the bottom surface 914 is coated with an adhesive to hold the monitoring device 900 to the skin. The four electrode pads 930, 932, 934, and 936 are connected to the skin through hydrogel patches within the adhesive. The battery housing 916 holds a coin type battery 938 that is mounted over the circuit board 920 as shown in FIG. 8C. In this example, the battery 938 is a non-rechargeable coin cell battery (e.g., a CR-2032 battery). Of course, other power sources such as a rechargeable battery may be used to power the monitor 900.

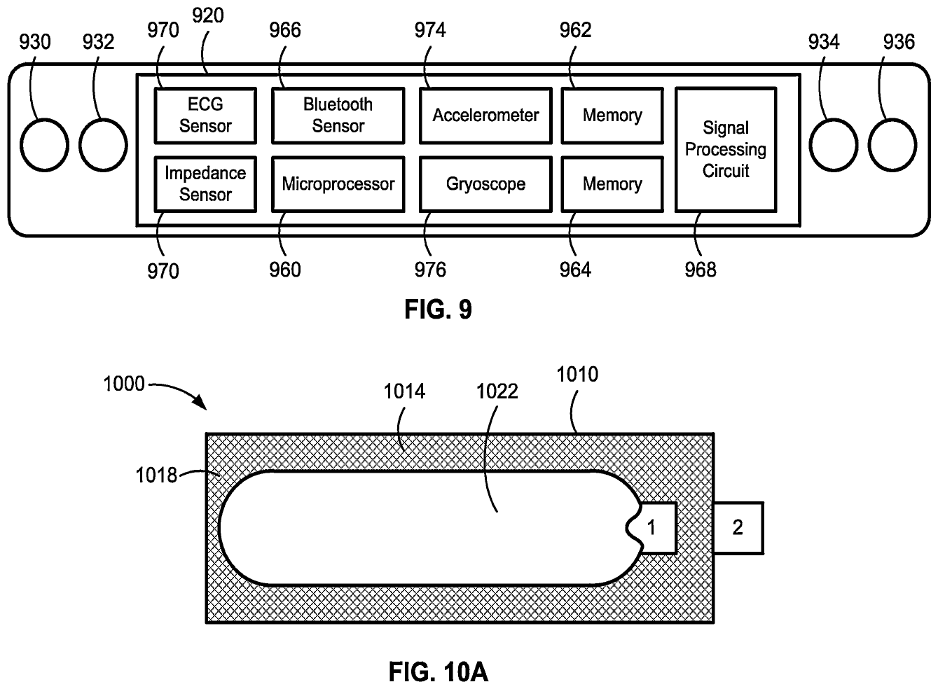

FIG. 9 is a block diagram of the electronic components of the example monitoring device 900. The monitoring device 900 includes a microprocessor 960, two writeable memories 962 and 964, a Bluetooth transceiver/antenna 966, and a signal processor circuit 968. The monitor 900 further includes an electrocardiogram (ECG) sensor 970, an impedance sensor 972, an accelerometer 974, and a gyroscope 976. The microprocessor 960 includes built in permanent memory that stores firmware for executing routines. Both of the memories 962 and 964 store data collected by the monitor 900. In this example, the memories allow storage of at least 80 hours of data. The collected data may be transmitted from the transceiver 966. Alternatively, a docking station may be provided that has connections to a computing device. The docking station includes contacts to charge a rechargeable battery as well as data contacts to allow data to be sent to the computing device.

In this example, the signal processor circuit 968 is an ASIC manufactured by MAXIM integrated (MAX30001) to measure ECG and chest impedance of the patient using signals received from the four electrode pads 930, 932, 934, and 936. In this example, the ECG sensor 970 is coupled to pads 932 and 934 to determine voltage signals for ECG. The impedance sensor 972 is coupled to the pads 932 and 934 to measure a voltage signal and to the pads 930 and 936 to inject low-amplitude (e.g. 92 microamps) high-frequency (e.g. 80 kHz) alternating current for determining impedance. The pads 932 and 934 are time-multiplexed between the ECG sensor 970 and the impedance sensor 972.

The data signals from the sensors 970 and 972, the accelerometer 974, and the gyroscope 976 are collected by the microprocessor 960. From this data, physiological signals such as heart rate, respiration rate, tidal volume, body position and body orientation may be extracted. The extracting or refining of data may be performed by the firmware on board the monitor 900 or on an external device such as a mobile device or a cloud-based server. As explained herein, the collected data may be used in the different processes to analyze health conditions of the patient. In this example, the collected physiological data may be used to determine tidal volume, respiration rate, minute ventilation, and tidal (as opposed to forced) breathing flow-volume curves and parameters derivable therefrom. The collected impedance values may be correlated with lung volume. The respiratory flow rate may be obtained from the time derivative of lung volume. Tidal volume parameters indicative of airway obstruction may be derived from a flow-volume curve constructed by plotting respiratory flow rate against lung volume.

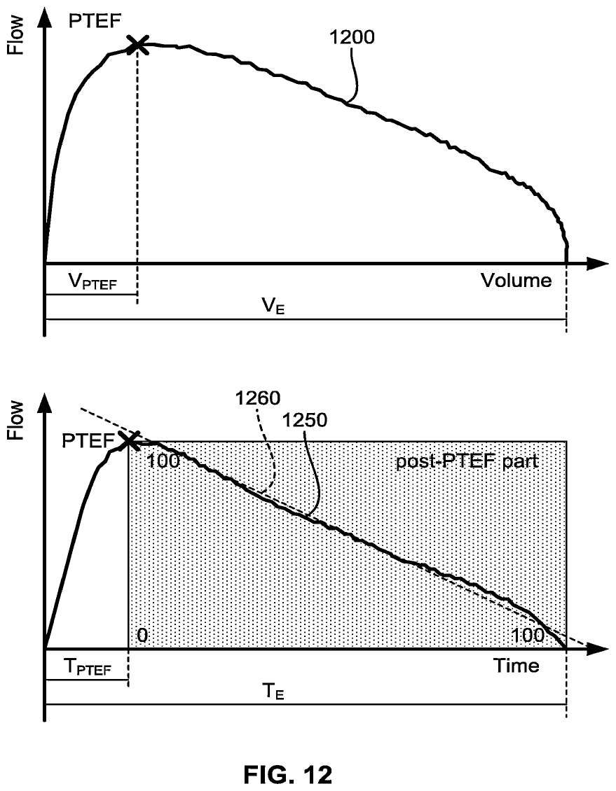

FIG. 12 contains two graphs illustrating a flow-volume curve and tidal volume parameters that may be extracted from such a curve. A trace 1200 in the upper graph represents a flow-volume curve constructed from data collected from a monitor 900 attached to a patient. A trace 1250 in the lower graph represents a profile of respiratory flow vs time, constructed from the same data as used to construct the flow-volume curve 1200. The flow-volume curve 1200 is constructed from the expiratory portion of the respiratory cycle such that positive values of respiratory flow rate (shortened to “flow” on the vertical axis label) represent expiratory flow, in keeping with the convention for spirometry. The profile 1250 likewise represents expiratory flow as positive on the vertical axis. The profile 1250 shows that the expiratory flow quickly increases to a peak value labelled as P_{TEF}, which is reached at the time labelled as T_{PTEF}, and thereafter decreases more slowly towards zero, which it reaches at the expiratory time labelled as T_{E}. A dashed line 1260 of slope S linearly approximates the post-peak decrease of expiratory flow. The flow-volume curve 1200 is traversed anti-clockwise, starting at the extreme right where lung volume is equal to the expiratory tidal volume V_{E}, and quickly reaching the peak expiratory flow value P_{TEF}, at which point the lung volume has decreased to V_{PTEF}, before falling gradually to the end of expiration where lung volume is defined to be zero.

Tidal volume parameters may be extracted from the traces 1200 and 1250. Three examples are:

Time to Peak Expiratory Flow over Expiratory Time (T_{PTEF}/T_{E})

Volume at Peak Expiratory Flow over Expiratory Tidal Volume (V_{PTEF}/V_{E})

Slope of post-peak Expiratory Flow Curve (S)

The tidal volume parameters, such as the three examples listed above, are indicative of the patient's respiratory condition and in particular of airway obstruction. In each example tidal volume parameter listed above, an increasing value is associated with bronchodilation, while a decreasing value is associated with bronchial obstruction. Other parameters, such as vital capacity, may also be derived from the physiological data. Some parameters may be derived that are capable of distinguishing between upper and lower airway obstruction in a way that conventional spirometry cannot do.

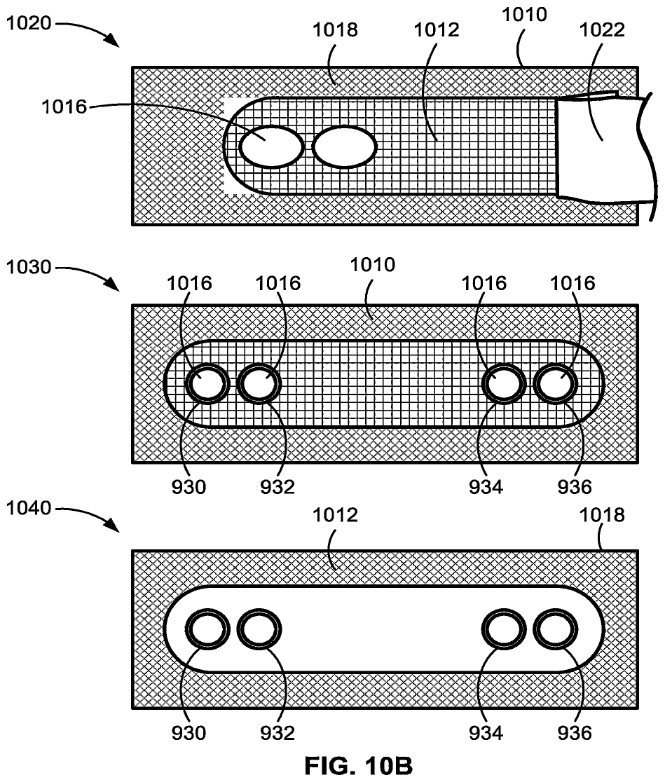

FIG. 10A is a perspective view of an example adhesive accessory 1000 for applying the example monitoring device 900 to the patient, prior to application. FIG. 10B shows successive steps in applying the adhesives in the adhesive accessory 1000 to monitoring device 900 before application to the skin of a patient. The adhesive accessory 1000 includes a protective bottom layer 1010 that supports a middle layer 1012 (shown in FIG. 10B). The middle layer 1012 has four hydrogels corresponding to the locations of the electrode pads 930, 932, 934, and 936 on the bottom surface 914 of the monitor 900 when properly attached to the middle layer 1012. A top protective layer 1014 comprising a skirt 1018 and a cutout portion 1022 in the shape of the monitor 900 covers the middle layer 1012.

As shown in a first step 1020 in FIG. 10B, a cutout 1022 of the top layer 1014 is peeled off to expose the middle layer 1012 with its hydrogels 1016 and their surrounding adhesives. The cutout 1022 is in the shape of the monitor 900, leaving the skirt 1018 of the top layer 1014 in place. As shown in step 1030 in FIG. 10B, which is an underside view of the adhesive accessory 1000, the monitor 900 is applied where the cutout 1022 was removed, becoming attached to the middle layer 1012 by the adhesives. The hydrogels 1016 on the middle layer 1012 thereby come into contact with the electrode pads 930, 932, 934, and 936 on the bottom surface 914 of the monitor 900, and are visible through the translucent bottom layer 1010. The bottom layer 1010 is then removed from the middle layer 1012 in step 1040, exposing the middle layer 1012 with its hydrogels 1016 and their surrounding adhesives. The middle layer 1012 with the now exposed adhesives and hydrogels 1016 is then attached via the adhesives to a suitable location on the chest of the patient so that the hydrogels 1016, and consequently the electrode pads 930, 932, 934, and 936, are in electrical contact with the skin. After the middle layer 1012 is successfully attached, the skirt 1018 of the top layer 1014 may be peeled off, leaving the monitor 900 and the middle layer 1012, which may be identified with the layer 918 in FIG. 8A, on the skin.

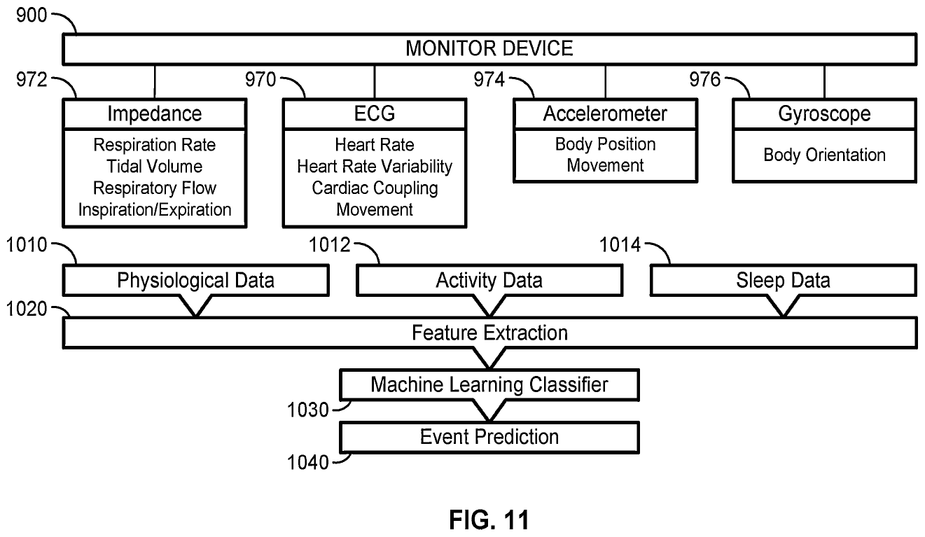

The monitor 900 may include other sensors such as an audio sensor. The monitor 900 may be used in place of the monitor 110 in the data collection and analysis process performed in the health care system 800 in FIG. 7. An example process flow of data collection from the monitor 900 for predictive analysis is shown in FIG. 11. Data is collected from sampling and correlating readings from the ECG sensor 970, impedance sensor 972, accelerometer 974 and gyroscope 976. The data is stored in the monitor 900 and repeatedly transmitted to an external device such as the portable device 112.

The collected data may be analyzed to create analytical data for predictive analysis. As shown in FIG. 11, the impedance data from the impedance sensor 972 may be used to determine respiration rate, tidal volume, respiratory flow rate, and inspiration/expiration. The ECG data from the ECG sensor 970 may be used to determine heart rate, heart rate variability, cardiac coupling and movement of the patient. The accelerometer data from the accelerometer 974 may be used to determine body position and bodily movement. The data from the gyroscope 976 may be used to determine body orientation.

The analyzed data from the sensors on the monitor 900 and optionally additional data from external sources may be classified into a set of physiological data 1110, a set of activity data 1112, and a set of sleep data 1114. The classified data is input into a feature extraction module 1120 that derives statistical features from these data such as mean, median, percentiles, standard deviation etc. This feature set is then input to a machine learning classifier 1130 that outputs an event prediction 1140. As described above in relation to FIG. 4, the event prediction 1140 may be the result of comparing a risk evaluation with a predetermined threshold.

The event prediction 1140 may be either a binary Yes/No indicator (event predicted/not predicted), or may be graded based on the severity of the predicted respiratory event such as mild, moderate and severe. In one implementation, the event prediction 1140 may be translated into different zones which may result in different corrective actions such as “time to take medication” or “seek medical advice from a health care professional” or “go to an emergency department”.

Additional outputs may include personalized medication reminders and dosage adjustments based on physiological data. Such reminders and adjustments according to personalized and dynamic medication therapy plans may be determined based on continuous monitoring of patient health status such as in the system shown in FIG. 1. Other functions for the collection of data may include:
helping clinicians in the diagnosis and management of asthma especially in children under the age of 5 who are unable to perform regular spirometry tests,
assessing the effectiveness of medication to ensure disease control or a need for a step-up or step-down medication usage and type,
helping with reducing the readmission rates of patients discharged from hospital following an acute asthma event.

A conventional medication therapy plan for asthma has two elements: a preventive medication element and a rescue medication element. The preventive medication element prescribes a certain dose (e.g., one puff) of a preventive medication (e.g., an anti-inflammatory) to be taken at regular intervals (e.g., once per day) regardless of symptoms. The rescue medication element prescribes a certain dose (e.g., one puff) of a rescue medication (e.g., a bronchodilator) to be taken in the event of symptoms occurring such as shortness of breath, wheezing etc., and subsequently at certain intervals, (e.g., four hours), if the symptoms have not abated. If symptoms have not abated after a certain number of doses of rescue medication, the plan calls for a visit to a doctor or a hospital.

One example of a personalized physiological signal may be specific pulmonary, cardiac, motion and other sensor readings captured from the other sensors in the monitor 110 in FIG. 1 or other monitors that may be attached to the patient 100. This data may be analyzed to provide a disease control and risk evaluation of the patient as described above. The determined risk evaluation may be used by a medication rules engine and dosage calculator executed by the data server 114 to provide personalized treatment to the patient. Such sensor readings may work with or without data gathered from connected dosing devices such as inhalers indicating whether a dose was delivered. In a similar manner, activities of the patient may also be monitored such as exercise or other physical activity. For example, this information can be used to assess if the respiratory condition is limiting the activity level of patient and if more medication is needed to bring normal activity level in patients.

The example medication rules engine and dosage calculator may be an application executed by a computing device such as the external portable device 112 or the server 114 in FIG. 1. The medication rules engine may include simple reminders and instructions for the patient or the family member of the patient for checking medication administration. Alternatively, the medication rules engine may use sensor data to determine that the medication was not taken or not taken properly. In one such example, this can be determined by matching the breathing profile with inhaler intake to ensure that an inhaler click was synchronized with inhalation. The medication effectiveness can be measured by comparing physiological data from pre-medication and with physiological data from post-medication. The medication rules engine may also provide instructions for an increased or decreased frequency of dosage, based on data from the sensors that provide the resulting effect, or lack thereof, the medication is having. Similarly, the medication rules engine may provide instructions to increase or decrease the dosage and/or type of medication (e.g. preventive, rescue, different drug, etc.) to address the effects, or lack thereof, of the current dosage.

The instructions to the patient or the family member of the patient may be done with or without the notice of a health care provider. For example, an OTC (over-the-counter) medication or a prescribed version of the medication may be approved for administration according to a medication rules engine that automatically adjusts dosage, within certain limits, without health care provider intervention. The medications may be administered by devices that provide any suitable drug delivery format, from inhalers to pills to drug-delivering patches. The medication rules engine may also incorporate patient reported symptoms such as shortness of breath, wheeze, cough, reduced activity and night-time awakening. In this example, the medication and medication rules engine may be specific to Asthma, COPD and other respiratory conditions, but other conditions may have other medication rules engines.

The same process could be employed in conjunction with other types of routines and plans that may be personalized, by contrast with current generalized and static plans. For example, such plans may include personalized and dynamic activity and exercise plans, personalized and dynamic cognitive and behavior plans, personalized and dynamic food and nutrition plans and personalized air-exposure plans. The repeated adjustment of such routines and plans provides such dynamic and personalized optimization. Aspects of the treatment, wellness and quality of life of the patient may be tailored to the individual patient and adapted to conditions of the patient and the environment. Causations of deviation from healthy status may also be analyzed. One example may be a patient having their own baseline and an adaptive algorithm that learns the individual thresholds for such a baseline. In this case deviations from a patient's own baseline can be of more concern than deviation from an age-matched heathy normal level.

The example analysis module executed by the data server 114 in FIG. 1 may also include population health factors in the asthma control and exacerbation prediction algorithms. The population health factors may provide more accurate predictions as respiratory ailments such as asthma are both local and seasonal. As explained in relation to the example in FIG. 1, physiological signals of interest are collected to determine symptoms and determine an individual's risk of falling out of asthma control or having an exacerbation such as an attack. Processing may take into account the patient's history/health record and any data on medication adherence, as captured through connected inhalers, for example, and environmental conditions (air quality including pollutants, allergens, etc.) based on geographic/home location-related data from third parties for each member of the general patient population. Such analysis may include the dynamic capture of local environmental data through indoor air quality monitors, or from outdoor sensors.

The analysis of exacerbation of respiratory ailments may also take into account population health factors that may be stored in the patient records in the database 250 in FIG. 2. Such factors may include social determinants of health (such as risk of food or housing insecurity, financial troubles, stress at home), as captured for each individual or calculated/inferred based on geographic/home location. In addition, the time of year may be used to further tune respiratory analysis. For example, there are known asthma spike times such as back to school time. The analysis may be used to stratify patients up front to quantify risks based on the variety of data described above.

The specific analysis in relation to a particular patient may be compared to the analysis of the general population or a specific cohort that is similar to the particular patient. For example, an individual patient may be dynamically grouped to other patients with similar socio-economic and ethnic traits. Any historical or new data gathered on others in the group may then be used to influence the prediction of a respiratory event for the individual patient. For example, shared EMR data on hospital admissions, health data (signs and symptoms), home addresses/Zip codes of admitted patients could be used to determine similar patient groups to improve predictions.

The monitoring experience may also be enhanced by providing incentives to both the patient and family members to adhere to the monitoring and any relevant treatment routines. This may be performed through gamification of the experience for both the patients and their family members. For example, child patients and their family members may receive points, badges, money, or other rewards for usage of a monitoring device such as the monitor 110 in FIG. 1. Such rewards may be obtained for wearing the monitor, charging the monitor, or taking the suggested therapy actions.

There could be teams of children and parents in competition against other teams for prizes such as an indoor air-quality monitor. Such a program may also bring in other partners (from government to private commercial or nonprofit) to contribute free/discounted services as the incentives. The incentives need not necessarily be directly asthma-related, but could be based on social determinants of health as above e.g. free meals or counselling. For example, the gamification application may offer free meals at participating healthy-food restaurants or the ability to make a donation when a patient completes a treatment or complies with a routine such as a workout. Adherence to a routine by wearing the monitor 110 could also provide the ability to donate to a cause, again made possible by a network of partners. The system may provide incentives to insurers/HMEs to take on populations of patients. For example, an insurer/HME may be credited with a donation to a health-related charity or other cause if they insure a certain population of patients. The incentives to patients, parents of patients, or other parties such as insurers may change based on changing social and environmental factors. For example, the rewards may increase when risks of non-adherence are higher. For example, on a sunny day, a certain reward may be offered for outside activity when pollutants/allergens are low. The reward would be reduced on high-pollutant days where risks of exacerbation are higher.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

Label list

| | |
|---|---|
| patient | 100 |
| monitor | 110 |
| portable device | 112 |
| data server | 114 |
| family member | 120 |
| alert device | 122 |
| environmental sensor | 130 |
| controller | 200 |
| sensor interface | 202 |
| transceiver | 204 |
| memory | 206 |
| battery | 208 |
| sensor | 210 |
| sensor | 212 |
| sensor | 214 |
| sensor | 216 |
| accelerometer | 218 |
| CPU | 230 |
| GPS receiver | 232 |
| transceiver | 234 |
| memory | 236 |
| application | 240 |
| data | 242 |
| database | 250 |
| analytics platform | 252 |
| machine learning module | 254 |
| step | 300 |
| step | 302 |
| step | 304 |
| step | 306 |
| step | 308 |
| step | 310 |
| step | 312 |
| step | 314 |
| step | 316 |
| step | 400 |
| step | 402 |
| step | 406 |
| step | 408 |
| step | 410 |
| step | 412 |
| step | 414 |
| early stage lung audio waveform | 500 |
| peaks | 502 |
| early stage heartbeat waveform | 510 |
| early stage respiratory waveform | 520 |
| late stage lung audio waveform | 530 |
| late stage heartbeat waveform | 540 |
| late stage respiratory waveform | 550 |
| example audio waveform | 560 |
| signatures | 562 |
| data | 570 |
| peaks | 572 |
| trace | 580 |
| trace | 582 |
| system | 600 |
| health care provider system | 610 |
| health care professional | 620 |
| supply system | 630 |
| system | 800 |
| EMR server | 814 |
| HCP server | 816 |
| patients | 820 |
| respective monitors | 822 |
| portable devices | 824 |
| wide area network | 830 |
| HCP server process | 854 |
| monitor | 900 |
| enclosure | 910 |
| top surface | 912 |
| bottom surface | 914 |
| battery housing | 916 |
| layer | 918 |
| circuit board | 920 |
| traces | 922 |
| electrode pad | 930 |
| electrode pad | 932 |
| electrode pad | 934 |
| electrode pad | 936 |
| battery | 938 |
| microprocessor | 960 |
| memory | 962 |
| memory | 964 |
| transceiver | 966 |
| signal processor circuit | 968 |
| ECG sensor | 970 |
| impedance sensor | 972 |
| accelerometer | 974 |
| gyroscope | 976 |
| adhesive accessory | 1000 |
| bottom layer | 1010 |
| middle layer | 1012 |
| top layer | 1014 |
| hydrogels | 1016 |
| skirt | 1018 |
| step | 1020 |
| cutout portion | 1022 |
| step | 1030 |
| step | 1040 |
| physiological data | 1110 |
| activity data | 1112 |
| sleep data | 1114 |
| feature extraction module | 1120 |
| machine learning classifier | 1130 |
| event prediction | 1140 |
| flow - volume curve | 1200 |
| profile | 1250 |
| dashed line | 1260 |

### REFERENCES

Seppä, V.-P., Pelkonen, A. S., Kotaniemi-Syrjänen, A., Mäkelä, M. J., Viik, J., & Malmberg, L. P. (2013). Tidal breathing flow measurement in awake young children by using impedance pneumography. J Appl Physiol, 1725-1731.

1-29. (canceled)

30. A continuous monitoring device attachable to a patient, the monitoring device comprising:
an enclosure having a surface that may be adhered to the patient;
a plurality of sensors, each of the plurality of sensors configured to output physiological data relating to a respiratory condition, disorder or ailment of the patient;
a memory configured to store the physiological data; and
a transceiver operable to transmit the physiological data to an external device.

31. The monitoring device of claim 30, wherein the plurality of sensors includes a heart rate sensor and a respiratory sensor.

32. (canceled)

33. The monitoring device of claim 32, further comprising a pair of electrode pads configured to sense a voltage between the electrode pads, wherein the heart rate sensor and the respiratory sensor are coupled to the pair of electrode pad.

34. (canceled)

35. (canceled)

36. The monitoring device of claim 33, further comprising a second pair of electrode pads to which the respiratory sensor is coupled for injection of low-amplitude, high-frequency current.

37. The monitoring device of claim 30, wherein the enclosure has a form factor that is one of the group consisting of: a patch, a wristband, a necklace, and a vest.

38. The monitoring device of claim 30, wherein the plurality of sensors includes at least one of an audio sensor, an accelerometer, a gyroscope or a pressure sensor.

39. (canceled)

40. (canceled)

41. The monitoring device of claim 30, wherein the enclosure is fabricated from a flexible compliant material.

42. A system to monitor a respiratory condition of a patient, the system comprising:
a monitor attachable to the patient, the monitor including:
a plurality of sensors, each of the plurality of sensors configured to output physiological data relating to the respiratory condition of the patient; and
a first transceiver configured to transmit the physiological data;
an external device including a second transceiver configured to receive the physiological data from the first transceiver; and
an analytics platform, coupled to the second transceiver, configured to:
analyze the physiological data received from the second transceiver to determine the occurrence of a symptom of the respiratory condition.

43. (canceled)

44. (canceled)

45. The system of claim 42, wherein the analytics platform is further configured to analyze environmental data related to the patient in determining the occurrence of the symptom of the respiratory condition.

46. The system of claim 42, wherein the analytics platform is further configured to analyze demographic data related to the patient in determining the occurrence of the symptom of the respiratory condition.

47. (canceled)

48. (canceled)

49. (canceled)

50. The system of claim 42, wherein the symptom is shortness of breath, wherein the plurality of sensors includes a pressure sensor, an accelerometer, and a respiratory sensor, and wherein the analytics platform is configured to determine shortness of breath using a combination of:
breathing effort determined from the pressure sensor and the accelerometer, and
respiration rate determined from the respiratory sensor.

51. (canceled)

52. The system of claim 42, wherein the plurality of sensors includes an audio sensor, and wherein the analytics platform is further configured to differentiate between a soft wheeze and other adventitious signals based on data from the audio sensor.

53. (canceled)

54. The system of claim 42, wherein the analytics platform is configured to apply a model to the physiological data to determine the occurrence of a symptom of the respiratory condition.

55. The system of claim 54, wherein the model is configured by machine learning based on collected physiological data and respiratory condition outcome data.

56. The system of claim 42, wherein the analytics platform is further configured to analyze the physiological data to determine a risk evaluation for a respiratory event of the respiratory condition.

57. (canceled)

58. (canceled)

59. (canceled)

60. The system of claim 56, wherein the plurality of sensors includes an impedance plethysmography sensor, and wherein the analytics platform is configured to determine the risk evaluation by:
correlating impedance measurements from the impedance plethysmography sensor with lung volume;
constructing a flow-volume curve from the lung volume;
extracting one or more tidal volume parameters from the flow-volume curve;
deriving features from the tidal volume parameters; and
applying a model to the features to determine the risk evaluation.

61. (canceled)

62. (canceled)

63. (canceled)

64. (canceled)

65. The system of claim 60, wherein the one or more tidal volume parameters are drawn from the group consisting of:
Time to Peak Expiratory Flow over Expiratory Time;
Volume at Peak Expiratory Flow over Expiratory Tidal Volume; and
Slope of post-peak Expiratory Flow Curve.

66. (canceled)

67. (canceled)

68. (canceled)

69. (canceled)

70. The system of claim 56, wherein the analytics platform is further configured to issue an alert based on the risk evaluation.

71. The system of claim 70, further comprising an alert device configured to:
receive the alert issued by the analytics platform, and
alert a person on receipt of the alert.

72-114. (canceled)

115. A method to monitor a respiratory condition of a patient, the method comprising:
transmitting physiological data from a monitor attached to the patient, the monitor including:
a plurality of sensors, each of the plurality of sensors configured to output physiological data relating to the respiratory condition of the patient; and
a first transceiver configured to transmit the physiological data;
receiving the physiological data on a second transceiver of an external device configured to receive the physiological data from the first transceiver; and
analyzing the physiological data received from the second transceiver via an analytics platform coupled to the second transceiver to determine the occurrence of a symptom of the respiratory condition.
